(19) United States
(12) Patent Application Publication
(10) Pub. No.: US 2007/0160691 A1
(43) Pub. Date: Jul. 12, 2007

(54) Pharmaceutical composition for treating and preventing cancer comprising cinnamoni cortex extract and zizyphi fructus extract

(75) Inventors: Byoung-Mog Kwon, Taejeon-si (KR); Kwang-Hee Son, Taejeon-si (KR); Dong Cho Han, Taejeon-si (KR); Yung-Hee Kho, Taejeon-si (KR); Hyo-Kon Chun, Taejeon-si (KR); Hwan Mook Kim, Taejeon-si (KR); Chang-Woo Lee, Taejeon-si (KR)

Correspondence Address: LUCAS & MERCANTI, LLP, 475 PARK AVENUE SOUTH, 15TH FLOOR, NEW YORK, NY, 10016, US

(73) Assignee: KOREA RESEARCH INSTITUTE OF BIOSCIENCE AND BIOTECH, Taejeon-si (KR)

(21) Appl. No.: 10/580,166

(22) PCT Filed: Dec. 23, 2004
(86) PCT No.: PCT/KR04/03426

§ 371 Date: Jun. 1, 2006

(30) Foreign Application Priority Data
Dec. 31, 2003 (KR) 10-2003-0101406

(51) Int. Cl. A61K 36/54; A61K 31/11; A61K 31/56

(52) U.S. Cl. 424/739; 514/169; 514/701

(57) ABSTRACT

Disclosed is a pharmaceutical composition for treating and preventing cancer, comprising a Cinnamoni Cortex extract and a Zizyphi Fructus extract. The pharmaceutical composition has effects of inducing apoptosis of cancer cells and inhibiting metastasis of cancer cells, as well as having a synergistic effect in inhibiting the growth of cancer cells compared to the separate use of the two extracts. Also, because the two extracts are extracted from natural origins, the pharmaceutical composition has no toxicity and is thus able to be used as a safe anticancer agent having excellent therapeutic efficacy. Further, the pharmaceutical composition is capable of being used as a functional food for preventing and treating cancer.

### TECHNICAL FIELD

The present invention relates to a pharmaceutical composition for treating and preventing cancer, comprising a Cinnamoni Cortex extract and a Zizyphi Fructus extract.

### BACKGROUND ART

The word cancer refers collectively to a family of diseases that starts mainly from the uncontrolled proliferation of cells, invades neighboring normal tissues or organs and establishes a new growth place therein and that can eventually take an individual's life. For the past ten years, conspicuous advances have been made in the regulation of cell cycle or apoptosis and in the development of novel targets including oncogenes and tumor suppressor genes. However, despite these efforts, the incidence of cancer is increasing as the world advances in cultivation.

At present, treatment of cancer patients is dependent on surgical operation, radiotherapy, and chemotherapy involving the administration of more than forty anticancer substances having strong cytotoxicity. Since these therapies are mostly limited to early-stage cancer patients or specific cancers, cancer death rates are increasing.

Chemotherapy involving administering anticancer substances has been successfully used in the treatment of testicular cancer or leukemia. However, chemotherapy is often not effective in the treatment of general epithelial-derived tumors, such as breast, colon and lung cancer. This is mainly due to the resistance of cancer cells to several anticancer agents, leading to very limited application of the anticancer agents.

Most currently available anticancer agents were developed based on the fact that cancer cells divide more rapidly than normal cells, and were exemplified by compounds inhibiting DNA replication or synthesis (cyclophosphamide, cisplatin, doxorubicin), metabolism inhibitors (methotrexate, 5-fluorouracil), cell division inhibitors (vincristine), nucleotide analogues (6-mercaptopurine) and topoismerase inhibitors (etoposide). Thus, anticancer agents typically affect the division and survival of cancer cells. As described above, since the conventional anticancer agents are focused on the rapid division of cancer cells, they are problematic in terms of having toxicity. A representative symptom is that body hair falls out. In addition, they have another disadvantage of affecting normal cells that rapidly divide under normal conditions, such as bone marrow cells and intestinal epithelial cells. Therefore, the development of anticancer agents having less toxicity is one of the problems to be solved in human society.

On the other hand, the effects of anticancer agents on cancer cells will be described with respect to three aspects: apoptosis of cancer cells, migration of cancer cells and cancer metastasis, as follows.

(1) Apoptosis

All cells of multicellular organisms have a potential to induce apoptosis, and homeostasis is maintained by the growth and death of cells. In an adult, fifty to seventy billion cells are removed daily by an apoptosis process, and a similar number of cells are generated, thus maintaining homeostasis. Cells undergoing apoptosis are absorbed via phagocytosis by surrounding cells. Under normal conditions, apoptosis involves the removal of cancer cells, the removal of self-activated leukocytes and tissue generation during development. However, since cancer cells have a defect in signal transduction associated with apoptosis, they increase in number and have resistance to anticancer agents.

Critical proteins involved in apoptosis have been conserved during animal evolution and have been targets for viruses. The evolutionary conserved proteins are key factors in apoptosis pathways, which include caspase/CED-3, Apaf-1/CED-4 and Bcl-2/CED-9.

Apoptosis is triggered mainly by two signaling pathways: one is mediated by cell death signal receptors presenting the plasma membrane, and the other by mitochondria present in cells.

Cell death receptors belong to the tumor necrosis factor receptor family that is characterized by having an intracellular death domain. The death domain binds to FADD (fas-associated death domain protein), and FADD then binds to inactive caspase-8 and caspase-10, resulting in caspase activation by self-cleavage.

Mitochondria-dependent apoptosis signaling, not clearly identified yet, is initiated by damaging mitochondria membrane and inducing the release of cytochrome c and other death factors from mitochondria. Cytochrome c in the cytosol binds to APAF1 (apoptotic protease activating factor-1), ATP and procaspase-9 to trigger the activation of caspase-9. Activated initiator caspases (caspase-8, -9 and -10) activate executioner caspases (caspase-3, -6 and -7). Activated caspases amplify cell death signaling by cleaving other inactive caspases. Eventually, activated executioner caspases degrade cell death substrates, resulting in the characteristic morphological and biochemical properties of dead cells. Representative examples of the celldeath substrates are as follows. The degradation of lamins induces nuclear shrinkage, and the degradation of PARP (poly(ADP-ribose) polymerase)results in the suppression of restoration of DNA damaged by external stresses, leading to cell death. In addition, cells shrink as cellular skeleton proteins such as actin are degraded, and are then removed by macrophages.

Intrinsic stresses, such as oncogenes, DNA damage, hypoxia and starvation, trigger the death of cells, and p53 is known as a critical regulator. p53 stimulates the expression of Bax, Bak, PUMA and Noxa, which promote apoptosis, while inhibiting the activity of a survival factor, Bcl-2, leading to the death of cells. Cancer cells often have defects in intrinsic apoptosis pathways. For example, in more than 50% of human cancers, the p53 suppressor gene is present in a mutated form and thus, the apoptosis of cancer cells is suppressed. Also, defects were found in downstream mediators of p53, such as PTEN, Bax, Bak and Apaf-1, and upstream regulators of p53, such as ATM, Chk2, Mdm2 and p19ARF, and these defects inhibit p53 from inducing apoptosis (Nature Review drug Discovery 2002, 1, 111-121).

(2) Migration of Cells

Cell migration is normal cell activity essential for the development and immunological defense of animals, and pathologically involves metastasis of cancer cells, wound healing, arteriosclerosis and inflammation. Metastasis of cancer cells has various stages involving metastasis, in which cancer cells pass through the extracellular matrix from an initial position and migrate into blood vessels, migration of cancer cells into the outside of blood vessels from a second target tissue, and migration of endothelial cells during angiogenesis.

Cell migration is a life phenomenon that is regulated by very complicated multistep processes, and is a very complex biological phenomenon that is mediated by numerous proteins in a number of steps and is regulated by numerous signal transducers. Representative proteins include integrin and cell growth factor receptors, which are known to be phosphorylated by kinases and control the growth, differentiation, death and migration of cells. Substances regulating cell migration have high potential to be developed as therapeutic agents against cancer and arthritis (Nature Review Molecular and Cell Biology, 2003, 4, 700-711).

(3) Cancer metastasis

Cancer is a leading cause of death due to its metastatic property. In fact, if an original site of cancer is identified, cancer can be easily removed by a simple surgical operation regardless of the original site. However, this surgical removal of cancer has a limitation when cancer cells metastasize to several sites other than a primary site, and thus, a complete cure of cancer by a surgical operation many be expected only in early stages. Since cancer cells grow uncontrollably and easily spread to neighboring organs, cancer is the most life-threatening disease.

Cancer metastasis is accomplished by a series of events, including steps in which metastatic cancer cells escape from an original site, invade surrounding tissues, and proliferate into secondary tumors in other sites. That is, the process of metastasis is composed of three major steps of migration, adhesion and invasion. At the first step, several matrix metalloproteins (MMPs) including MMP-2 (matrix metalloproteinase-2) play an critical role in that cancer cells degrade the extracellular matrix (ECM) and the basement membrane (BM) to trigger their invasion into surrounding tissues. MMPs, a group of secretory or trans membrane enzymes participating in the degradation of components of the ECM and BM, are divided into four groups according to their structural and functional properties: collagenases degrading BM collagen, stromelysins degrading proteoglycans and glycoproteins, gelatinases degradging BM collagen and gelatin, and membrane-type MMPs (MT-MMPs). Recently, 17 types of MMPs, including collagenase-3 and four MT-MMPs, have been isolated. In particular, among these MMPs, MMP-2 and MMP-9, which degrade a major component of the BM, type IV collagen, are known to be excessively secreted in highly metastatic cancer cells, such as B16-F10. With this evaluation, that MMP-2 and MMP-9 are important target enzymes toward the inhibition of cancer metastasis, many studies are focused on the development of inhibitors of these enzymes (Nature Review Cancer 2001, 1, 46-54).

### DISCLOSURE OF INVENTION

### Technical Solution

Therefore, the present invention aims to provide a pharmaceutical can position and a functional food, each of which comprises natural extracts having an effect of inhibiting the growth of cancer cells with no toxicity.

### Advantageous Effects

The mixture of the Cinnamni Cortex extract and the Zizyphi Fructus extract according to the present invention has effects of inducing apoptosis of cancer cells and inhibiting cancer metastasis, as well as being more effective in inhibiting the growth of cancer cells than when used separately. Also, the extracts have no toxicity and are thus capable of being used as a functional food for preventing and treating cancer.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a photograph showing the results of electrophoresis analysis, indicating that DNA cleavage is induced by a compound represented by Chemical Formula 1 according to the present invention;

FIG. 2 is a photograph showing the results of Western blotting, indicating that PARP (poly(ADP-ribose) polymerase) cleavage is induced by a compound represented by Chemical Formula 1 according to the present invention; and

FIG. 3 is a graph showing the inhibitory effect of a compound represented by Chemical Formula 1 according to the present invention regarding the migration of cancer cells.

### BEST MODE FOR CARRYING OUT THE INVENTION

To accomplish the above object, the present invention provides a pharmaceutical composition for preventing and treating cancer, comprising a Cinnamoni Cortex extract and a Zizyphi Fructus extract.

Hereinafter, the present invention will be described in detail.

The present invention provides a pharmaceutical composition for preventing and treating cancer, comprising a Cinnamoni Cortex extract including a compound represented by Chemical Formula 1, below, and a Zizyphi Fructus extract including a compound represented by Chemical Formula 2, below.

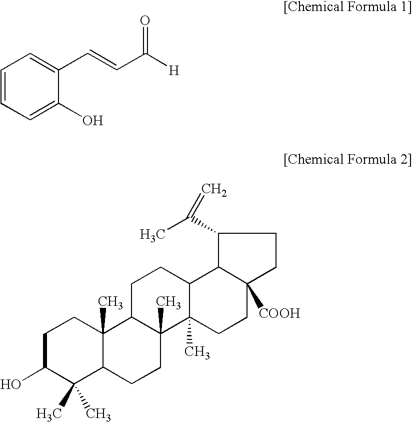

The Cinnammni Cortex extract is prepared by extracting finely cut Cinnamoni Cortex with ethanol, adding a basic aqueous solution to the resulting extract to form a salt, extracting the resulting solution with ethyl acetate, and concentrating the resulting extract under pressure. The basic aqueous solution is exemplified by sodium hydroxide. The formed salt is neutralized using an acidic aqueous solution. The obtained Cinnamni Cortex extract contains more than about 50% of 2′-hydroxycinnamaldehyde (HCA) of Chemical Formula 1, above. The Cinnamoni Cortex extract induces apoptosis in most cancer cells, including colon cancer and breast cancer cells, and inhibits the migration of cancer cells, as well as being nontoxic and thus safe because it is obtained from a naturally occurring source.

The Zizyphi Fructus extract is prepared by reflux-extracting finely cut flesh of Zizyphi Fructus with methanol, adding a basic aqueous solution to the resulting extract to form a salt, extracting the resulting solution with ethyl acetate, and concentrating the resulting extract under pressure. The basic aqueous solution is exemplified by sodium hydroxide. The formed salt is neutralized using an acidic aqueous solution. The obtained Zizyphi Fructus extract contains more than 50% betulinic acid (BTA) of Chemical Formula 2, above. The Zizyphi Fructus extract is effective in inhibiting metastasis of cancer cells, and is nontoxic and thus safe because it is obtained from a naturally occurring source.

The present invention is characterized by employing a mixture of the Cinnamoni Cortex extract and the Zizyphi Fructus extract as an anticancer agent. As noted above, the Cinnamoni Cortex and Zizyphi Fructus extracts have individually anticancer activity. Also, when used together, the extracts have effects of inducing apoptosis of cancer cells and inhibiting cancer metastasis, as well as being more effective in inhibiting the growth of cancer cells than when used separately. Further, the extracts have no toxicity and are thus able to be used as safe anticancer agents.

The Cinnamoni Cortex extract is mixed with the Zizyphi Fructus extract in a ratio of 20:80 to 80:20 by wt %. Within this range, the extracts have synergistic effects. When the extracts are mixed outside this range, the synergistic effects of the combination of the extracts are not achieved.

That is, upon clinical application, the pharmaceutical composition of the present invention many be administered orally or parenterally in various formulations. The pharmaceutical composition may formulated into a pharmaceutical preparation using a filler, a thickening agent, a binder, a humectant, a disintegrator, a diluent such as a surfactant, or an excipient. Examples of solid preparations for oral administration include tablets, pills, powders, granules, capsules and troches. Such solid preparations may be prepared by mixing the compound of Chemical Formula 1 with at least one excipient, for example, starch, calcium carbonate, sucrose or lactose, and gelatin. In addition to the simple excipient, the solid preparations many also contain a lubricant such as magnesium stearate or talc. Examples of liquid preparations for oral administration include suspensions, liquid solutions, emulsions and syrups. The liquid preparations may contain a simple diluent such as water or liquid paraffin, and various excipients, for example, humectants, sweetening agents, aromatics and preservatives. Examples of pharmaceutical preparations for parenteral administration include sterilized aqueous solutions, non-liquid solutions, suspensions, emulsions, freeze-dried preparations and suppositories. The non-liquid solutions and suspensions may be prepared using propylene glycol, polyethylene glycol, vegetable oils such as olive oil, and injectable esters such as ethyloleate. Bases for the suppositories may include Witepsol, Macrogol, Tween-61, cacao oil, laurin fat, glycerol and gelatin.

In addition, human dosage of the pharmaceutical composition of the present invention may vary depending on the patient's age, weight, gender, state of health and severity of the illness, and administration forms, and is typically 100-1000 mg/day, and preferably, 100-500 mg/day, based on an adult patient weighing 70 kg. Also, the pharmaceutical composition may be administered once to several times per day at predetermined-determined intervals according to the judgment of a doctor or pharmacist.

In addition, the present invention provides a functional food for preventing and treating cancer, comprising a mixture of the Cinnamoni Cortex extract and the Zizyphi Fructus extract as an effective component.

As described above, the mixture of the Cinnamoni Cortex extract and the Zizyphi Fructus extract according to the present invention has effects of inducing apoptosis of cancer cells and inhibiting cancer metastasis, as well as being more effective in inhibiting the growth of cancer cells than when used separately. Also, the extracts have no toxicity and are thus capable of being used as a functional food for preventing and treating cancer.

The functional food of the present invention may be prepared in the form of general foods, such as suspensions and beverages. The functional food of the present invention may contain any general food additive, and may be manufactured by adding food additives to the present mixture of the extracts having anticancer activity. Herein, the content of the food additives may be determined suitably for the taste, texture and preference of consumers, and is preferably 0.001-10 wt % based on the total weight of the functional food.

In addition, the functional food of the present invention may be provided in the form of beverages or suspensions, and may include, in addition to the mixture of the extracts, ingredients typically added to beverages, which are selected from the group consisting of vitamin C, vitamin E powder, iron lactate, zinc oxide, nicotinic acid amide, vitamin A, vitamin B₁ vitamin B₂ and mixtures thereof.

A better understanding of the present invention may be obtained through the following examples which are set forth to illustrate, but are not to be construed as the limit of the present invention.

### PREPARATIONAL EXAMPLE 1

### Preparation of a Cinnamoni Cortex Extract

1 kg of Cinnamoni Cortex, purchased from a store selling Chinese herbal medicines, was finely cut, extracted with 100% hexane for 24 hrs, and filtered to remove oily components.

The Cinnamoni Cortex, from which oily components had been removed, was extracted with 70% ethanol for three days and filtered to provide an ethanol extract.

The ethanol extract was concentrated in a water bath at 60° C. under pressure. The resulting concentrate was suspended in 2 L of distilled water, supplemented with a suitable amount of 0.1 N sodium hydroxide to form a salt, and supplemented with 2 L of ethyl acetate to separate an aqueous layer. Using a separatory funnel, a soluble part was eliminated. The resulting solution was neutralized with 0.1 N HCl, and supplemented and extracted with 2 L of ethyl acetate while being shaken. The resulting extract was concentrated in a water bath at 40° C. under pressure, thus producing a Cinnamoni Cortex extract.

The Cinnamoni Cortex extract was subjected to ODS column chromtography using an 80% methanol solution as an elution solvent. As a result, the Cinnamoni Cortex extract was found to contain more than 50% HCA as an active component.

### PREPARATIONAL EXAMPLE 2

### Preparation of a Zizyphi Fructus Extract

1 kg of flesh of Zizyphi Fructus purchased from a store selling Chinese herbal medicines was finely cut, reflux-extracted with 100% methanol for 24 hrs and filtered under pressure. The filtrate was concentrated in a water bath at 60° C. under pressure. The resulting methanol extract was suspended in 2 L of distilled water. The methanol extract suspended in distilled water was supplemented with a suitable amount of 0.1 N sodium hydroxide to form a salt of an acidic compound, and supplemented and extracted with 2 L of ethyl acetate while being shaken to eliminate an ethyl acetate-soluble part. The resulting solution was neutralized with 0.1 N HCl, and supplemented and extracted with 2 L of ethyl acetate while being shaken. The resulting extract was concentrated in a water bath at 60° C. under pressure, thus producing a Zizyphi Fructus extract.

The ethyl acetate extract was dissolved in 40 ml methanol and fractionated using a reverse-phased silica gel, thus generating a 60% acetonitrile fraction and a 70% acetonitrile fraction. The fractions were subjected to ODS column chromatography using a 70% acetonitrile solution as an elution solvent. As a result, the Zizyphi Fructusextract was found to contain more than 50% betulinic acid (BTA) as an active component.

### EXAMPLE 1

### Preparation of an Extract Mixture According to the Present Invention

An extract mixture according to the present invention was prepared by mixing 50 mg of the Cinnamoni Cortex extract prepared in Preparational Example 1 with 50 mg of the Zizyphi Fructus extract while stirring at roan temperature.

### EXPERIMENTAL EXAMPLE 1

### Evaluation of the Effect of HCA on the Induction of Apoptosis of Cancer Cells

MDA-MB-231 breast cancer cells and SW620 colon cancer cells were seeded onto 100-mm culture dishes at densities of 1×10⁶ and 2×10⁶ cells, respectively. After 18 hrs, the cells were treated with 10 μM and 30 μM of 2′-hydroxycinnamaldehyde(HCA) and further cultured for 48 hrs.

Then, to analyze DNA cleavage by the HCA treatment, the cells were lysed using RIPA buffer (50 mM Tris-HCl, pH 7.4, 150 mM NaCl, 5 mM EDTA, 1% NP-40, 1 mM sodium vanadate, 0.5% sodium deoxycholate, and 0.05% sodium deoxysulfate). The cell lysates were centrifuged at 13,000 rpm. The supernatants were subjected to phenol extraction to remove proteins, and the nucleic acid layers were recovered. Potassium acetate was added to each nucleic acid layer in a final concentration of 50 mM, and an equal volume of isopropyl alcohol was added thereto to precipitate nucleic acids. The precipitated nucleic acids were washed with 80% ethanol and dried. The dried nucleic acids were dissolved in 30 μl of TE buffer, and treated with Dnase-free RNase at 37° C. for 1 hr to remove RNA.

The resulting DNA samples were electrophoresed on a 1.5% agarose gel to investigate DNA fragmentation by HCA.

The results are given in FIG. 1.

As shown in FIG. 1, compared to cells treated with DMSO, HCA-treated MDA-MB-231 and SW620 cells displayed fragmentation of chromosonal DNA into uniform DNA fragments. This DNA fragmentation, one of the characteristic features of apoptosis, indicates that HCA induces apoptosis of cancer cells. UV was used as a positive control. When cells were exposed to UV for 15 min, UV caused damage on chromosomal DNA, resulting in apoptosis induction.

These results indicate that HCA induce apoptosis of cancer cells.

### EXPERIMENTAL EXAMPLE 2

### Evaluation of the Effect of HCA on PARP Cleavage

MDA-MB-231 breast cancer cells and SW620 colon cancer cells were seeded onto 100-mm culture dishes at densities of 1×10⁶ and 2×10⁶ cells, respectively. After 18 hrs, the cells were treated with 10 μM and 30 μM of HCA and further cultured for 48 hrs.

Then, to analyze PARP (poly(ADP-ribose) polymerase) cleavage by the HCA treatment, the cells were lysed using RIPA buffer (50 mM Tris-HCl, pH 7.4, 150 mM NaCl, 5 mM EDTA, 1% NP-40, 1 mM sodium vanadate, 0.5% sodium deoxycholate, and 0.05% sodium deoxysulfate). The cell lysates were centrifuged at 13,000 rpm and the supernatants were recovered. In the supernatants, protein concentrations were determined using a Bradford reagent (Bio-Rad Protein Assay, USA). Protein samples of 30 μg were separated on a 7.5% SDS-PAGE (SDS-polyacrylamide gel electrophoresis) gel. Proteins were then electro-transferred onto a PVDF membrane. The blot was blocked using 5% skim milk in TBST (50 mM Tris-HCl, pH 7.6, 150 mM NaCl, 0.1% Tween 20). Intracellular PARP was detected using a PARP-specific antibody (Cell signaling Technology, USA), a HRP (horse radish peroxidase)-conjugated secondary antibody, and a chemiluminescence POD reagent (Roche, Germany).

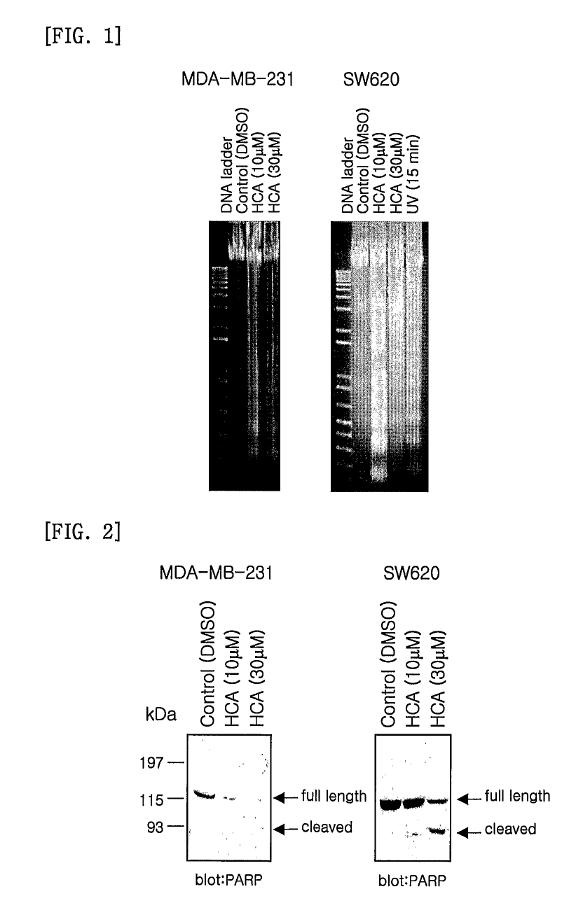

The results are given in FIG. 2.

As shown in FIG. 2, compared to cells treated with DMSO, HCA-treated MDA-MB-231 and SW620 cells displayed a decrease in normal full-length PARP (116 kDa) and an increase in cleaved PARP (89 kDa). PARP is an enzyme involved in the repair of damaged DNA and, during apoptosis, is cleaved by caspase-3. Thus, these results indicate that HCA induces caspase-3-mediated apoptosis in cancer cells.

### EXPERIMENTAL EXAMPLE 3

### Evaluation of the Inhibitory Effect of HCA Against the Migration of Cancer Cells

Cultured MDA-MB-231 breast cancer cells were detached from culture dishes using a trypsin/EDTA solution (Gifco, USA), neutralized with trypsin inhibitor (0.5 mg/ml, Sigma, USA), and washed twice with culture medium. The cells were then counted using a hematocytometer. Cell migration was assayed using a Boyden chamber. HCA was added to a lower compartment of the Boyden chamber through holes with concentrations of 0 μM, 1 μM, 5 μM and 10 μM. After the lower compartment of the Boyden chamber was covered with a polycarbonate membrane (8 μm pore, Neuro Probe, Inc, USA), an upper compartment of the Boyden chamber was assembled with the lower compartment, and 6×10 MDA-MB-231 cells were inoculated in the upper compartment through holes. After the Boyden chamber was incubated in an incubator for 6 hrs, the membrane was immersed in methanol to fix the cells. Then, the membrane was air-dried, stained with a 10% Girkmsa staining solution (Sigma, USA)for 1 hr, and destained with water for 10 sec. Herein, after non-migratedcells (an upper side of the membrane) were removed using a swab, migrated cells (a lower side of the membrane) were counted under a microscope.

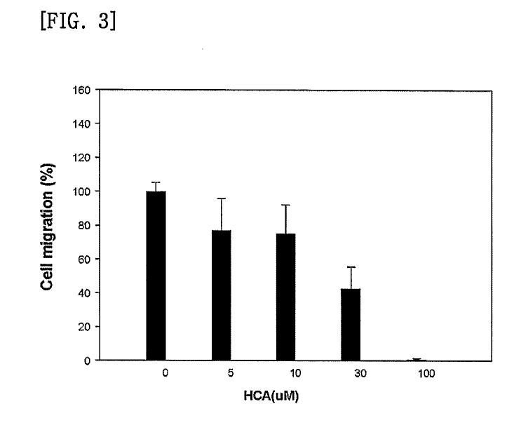

The results are given in FIG. 3.

As shown in FIG. 3, HCA inhibited cell migration even at 5 μM and, at 30 μM, inhibited more than 50% of cell migration.

### EXPERIMENTAL EXAMPLE 4

### Evaluation of the Inhibitory Effect of the Zizyphi Fructus Extract Against Lung Metastasis of B16-F10 Melanoma Cells

B16-F10 melanoma cells, subcultured in RPMI1640 medium containing 4% fetal bovine serum (FBS), were washed twice with physiological saline pre-cooled on ice. Then, 0.2 ml of a cell suspension, corresponding to a lethal dose of 5×10⁶ cells/mouse, was injected into the tail veins of nice. A sample to be tested was dissolved in injectable saline, and administered in a dose of 0.2 ml per day immediately after the melanoma cell transplantation. A partially purified extract was orally administered fifteen times in a dose of 100 mg/kg/day, and a control group was injected with 0.2 ml of injectable saline. Anticancer activity of the sample was assayed in the lung of the nice. That is, 15 days after the melanoma cell transplantation, an incision was made on the breast of each mouse, and the lung was excised from the mouse and tumors formed were analyzed.

As a result, when nice were orally administered with the Zizyphi Fructus extract containing betulinic acid as a major component in a dose of 100 mg/kg/day, lung metastasis of melanoma cells was inhibited by 60% in comparison with the control group having a melanoma lung metastasis rate of 100%.

### EXPERIMENTAL EXAMPLE 5

### Evaluation of the Anticancer Effect of a Mixture of the Cinnamoni Cortex and Zizyphi Fructus Extracts

Samples were prepared using 0.5% Tween 80 as a median, which included 0.5% Tween 80 as a control, the Cinnamoni Cortex extract (66 mg/kg) and a mixture (100 mg/kg) of the Cinnamoni Cortex and Zizyphi Fructus extracts. SW620 human colon cancer cells were used as a cancer cell line, and 0.3 ml/mouse of a suspension of the cancer cells (1×10⁷ cells/ml) was subcutaneously transplanted. From Day 1 to Day 21 after the cancer cell transplantation, each sample was orally administered once per day in a dose of 10 ml/kg. From Day 8 to Day 22 after the cancer cell transplantation, tumor size was measured a total of six times in each mouse. Weight change was measured a total of eight times from the starting day to the ending day. On Day 22 after the cancer cell transplantation, nude mice were sacrificed, and tumor were isolated from the nice and weighed.

When the cancer cell-transplanted nude mice were orally administered with each sample everyday for a period of 21 days, no weight loss was observed in all samples. However, with respect to tumor size, when the Cinnamoni Cortex extract was singly administered, it showed an effect of inhibiting the growth of cancer cells by 32.6% (p<0.05) in comparison with a control group. Also, the mixture of Cinnamoni Cortex and Zizyphi Fructus extracts was found to have an effect of inhibiting the growth of cancer cells by 51.8% (p<0.01). When SW620 tumors were excised and weighed on Day 22 after the tumor transplantation, the single administration of the Cinnamoni Cortex extract and the combinational administration of the Cinnamoni Cortex and Zizyphi Fructus extracts were found to significantly reduce tumor size by 26.1% (p<0.05) and 46.2% (p<0.01), respectively.

### EXPERIMENTAL EXAMPLE 6

### Evaluation of the Acute Toxicity of the Cinnamoni Cortex Extract and the Zizyphi Fructus Extract in Rats Upon Oral Administration

The Cinnamoni Cortex and Zizyphi Fructus extracts according to the present invention were evaluated for acute toxicity, as follows.

An acute toxicity assay was carried out using six week-old specific pathogen-free (SPF) SD rats. Two groups, each of which consists of two rats, were orally administered once with the Cinnamoni Cortex extract and the Zizyphi Fructus extract, respectively, which were suspended in a 0.5% methylcellulose solution. Thereafter, mortality, clinical symptoms and weight change were examined, and hematological and blood biochemical tests were carried out. Also, the rats were evaluated for abnormality in abdominal and thoracic organs by autopsy.

As a result, all of the rats administered with the extracts showed no specific clinical symptoms, no death and no weight change. Also, the hematological test, blood biochemical test and autopsy revealed that the two extracts have no toxicity. The Cinnamoni Cortex and Zizyphi Fructus extracts according to the present invention displayed no toxicity even at a dose of 1000 mg/kg and were found to have a minimum lethal dose (LD ) greater than 1000 mg/kg upon oral administration, thereby indicating that they are safe.

### FORMULATION EXAMPLE 1

### Preparation of Capsules

10 mg of the mixture of the Cinnamoni Cortex and Zizyphi Fructus extracts according to the present invention, prepared in Example 1, was mixed with 14.8 mg of lactose, 10.0 mg of polyvinylpyrrolidone and 0.2 mg of magnesium stearate. The resulting mixture was filled into No. 5 gelatin capsules using a suitable apparatus.

Contents of the capsules are as follows.

The extract mixture of Example 1 10.0 mg

Lactose 14.8 mg

Polyvinylpyrrolidone 10.0 mg

Magnesium stearate 0.2 mg

### FORMULATION EXAMPLE 2

### Preparation of an Injectable Solution

10 mg of the mixture of the Cinnamoni Cortex and Zizyphi Fructus extracts according to the present invention, prepared in Example 1, was mixed with 180 mg of mannitol, 26 mg of Na₂HPO₄.12H₂O and 2974 mg of distilled water. The resulting solution was added to a bottle, incubated at 20° C. for 30 min and sterilized, thereby generating an injectable solution.

The composition of the injectable solution is as follows.

The extract mixture of Example 1 10.0 mg

Mannitol 180 mg

Na2HPO₄.12H₂O 0 26 mg

Distilled water 2974 mg

### FORMULATION EXAMPLE 3

### Preparation of a Beverage

The extract mixture of the present invention, prepared in Example 1, was thoroughly mixed with vitamin C, vitamin E powder, iron lactate, zinc oxide, nicotinic acid amide, vitamin A, vitamin B₁ and vitamin B₂, thus generating a beverage.

The composition of the beverage is as follows.

The extract mixture of Example 1 0.1 g

Vitamin C 15 g

Vitamin E powder 7.5 g

Iron lactate 19.75 g

Zinc oxide 3.5 g

Nicotinic acid amide 3.5 g

Vitamin A 0.2 g

Vitamin B₁ 0.25 g

Vitamin B₂ 0.3 g

### INDUSTRIAL APPLICABILITY

As described hereinbefore, the present composition comprising a mixture of Cinnamoni Cortex and Zizyphi Fructus extracts has effects of inducing apoptosis of cancer cells and inhibiting metastasis of cancer cells due to its components, as well as having a synergistic effect in inhibiting the growth of cancer cells compared to the separate use of the two extracts. Also, because the two extracts are extracted from natural origins, the present composition has no toxicity and is thus able to be used as a safe anticanceragent having excellent therapeutic efficacy. Further, the present composition is capable of being used as a functional food for preventing and treating cancer

1. A pharmaceutical composition for preventing and treating cancer, comprising a Cinnamoni Cortex extract including a compound represented by Chemical Formula 1, below, and a Zizyphi Fructus extract including a compound represented by Chemical Formula 2, below.

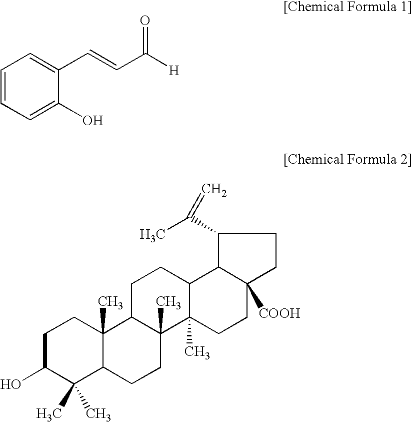

2. The pharmaceutical composition according to claim 1, wherein the Cinnamoni Cortex extract is prepared by extracting finely cut Cinnamoni Cortex with ethanol, adding a basic aqueous solution to a resulting extract to form a salt, extracting a resulting solution with ethyl acetate, and concentrating a resulting extract under pressure.

3. The pharmaceutical composition according to claim 1, wherein the Zizyphi Fructus extract is prepared by reflux-extracting finely cut flesh of Zizyphi Fructus with methanol, adding a basic aqueous solution to a resulting extract to form a salt, extracting a resulting solution with ethyl acetate, and concentrating a resulting extract under pressure.

4. The pharmaceutical composition according to claim 1, wherein the Cinnamoni Cortex extract is mixed with the Zizyphi Fructus extract in a ratio of 20:80 to 80:20 by wt %.

5. The pharmaceutical composition according to claim 1, wherein the composition has effects of inhibiting growth of cancer cells, inducing apoptosis of cancer cells and inhibiting cancer metastasis.

6. A functional food for preventing and treating cancer, comprising the mixture of the Cinnamoni Cortex extract and the Zizyphi Fructus extract according to claim 1 as an effective component.
